SEQUENCE LISTING (1 sequence)

SEQ ID NO: 1 - DNA, 11 bp, Artificial Sequence
  synthetically constructed poly T/U
uttuttutuuu

(19) United States
(12) Patent Application Publication
(10) Pub. No.: US 2015/0037357 A1
(43) Pub. Date: Feb. 5, 2015

(54) IMMUNE STIMULATION/METABOLIC INHIBITION AS ANTITUMOR THERAPY

(71) Applicant: NovaRx Corporation, San Diego, CA (US)

(72) Inventors: Habib Fakhrai, La Jolla, CA (US); Daniel L. Shawler, San Diego, CA (US)

(21) Appl. No.: 14/368,277

(22) PCT Filed: Dec. 21, 2012
(86) PCT No.: PCT/US2012/071316

§ 371 Date: Jun. 23, 2014

Related U.S. Application Data
(60) Provisional application No. 61/580,153, filed on Dec. 23, 2011.

(51) Int. Cl. A61K 39/00; A61K 39/395; A61K 31/7088

(52) U.S. Cl. CPC A61K 39/0011; A61K 31/7088; A61K 39/39558; A61K 2039/505; A61K 2039/585
USPC 424/174.1; 424/277.1; 536/23.1; 424/178.1; 530/391.7

(57) ABSTRACT

Methods, compositions and compounds that include a nucleic acid comprising a sequence consisting essentially of polyT/polyU and an agent that inhibits the production or activity of an immunosuppressive compound for treating tumors are described.

### CROSS-REFERENCE TO RELATED APPLICATION

This application claims priority from U.S. Ser. No. 61/580,153 filed Dec. 23, 2011. The contents of this application is hereby incorporated by reference herein in its entirety.

### TECHNICAL FIELD

The invention concerns a new approach to antitumor therapy. More specifically, it concerns methods and compositions that both stimulate the immune system by dampening endogenous immunosuppression by, and suppress the metabolism of, tumor cells.

### BACKGROUND ART

The treatment of malignancy has often employed agents that are nonspecific to a particular type of tumor, such as chemotherapy and radiation, as well as surgery. None of these methods is particularly attractive, as the side effects of all of these approaches range from uncomfortable to physiologically and psychologically devastating. The need for a generic treatment lacking these side effects is therefore intense. The present invention provides such a treatment.

US2010/0144831 describes, among other therapeutic methods, a method to inhibit tumor metabolism by administering to the tumor a single-stranded antisense nucleic acid or a modified form thereof having a sequence consisting of a polyT or polyU that is complementary to the mRNA polyA tail, or administering a vector that will generate a polyU RNA molecule. According to this disclosure, these constructs are conveniently, although not necessarily, provided intratumorally. The description set forth in the '831 document is incorporated herein by reference in its entirety. Particular cancers that are targets of such treatment are illustrated on paragraph 40 of this document. These are illustrative only, since the effectiveness of these compositions is not dependent on the type of cancer. As pointed out in paragraph 70 thereof, because the polyA tail is a universal characteristic of eukaryotic mRNA it is almost impossible for the tumor to develop a rescue mechanism against this treatment.

WO96/02143 published 1 Feb. 1996 describes administering to subjects tumor cells that are similar to a subject's cancer cells, but that are genetically modified to reduce or inhibit the expression of one or more immunosuppressive agents produced by the administered cells. A description of such immunosuppressive agents is provided and methods to inhibit their expression is also described. As noted, this document suggests inhibiting expression of such immunosuppressive agents only in allogeneic cells administered as antitumor vaccines. There is no suggestion to inhibit expression or activity of such agents that are present in the subject's tumor cells themselves. The contents of this document are also incorporated herein by reference.

The present invention provides compositions that are effective both in shutting down the metabolism of tumor cells and in inhibiting the endogenous tumor cells from exhibiting immunosuppression.

### DISCLOSURE OF THE INVENTION

The invention provides antitumor treatments that contain, as active ingredients, nucleic acid molecules or modified forms thereof that contain, polyT or polyU sequences and are thus designed to bind exclusively to polyA tails of mRNA as well as one or more agents that inhibit the expression or activity of an immunosuppressive molecule produced by the endogenous tumor cells. As noted above, because the polyA tail is common to essentially all mRNA's, it is impossible for tumor cells to develop resistance to the inhibition of expression effected by these compositions. However, because many cancers are prevented or treated endogenously by the subject's own immune system, many cancers produce immunosuppressive agents as protectors against immune surveillance.

The components of the treatment may be present as separate molecular entities, or may be covalently or noncovalently associated. Covalently coupled polyU/polyT containing molecules coupled to inhibitors of expression or activity of immunosuppressive agents are themselves novel and are also the subject of the present invention.

Thus, in one aspect, the invention is directed a method to treat a tumor or tumors in a subject which method comprises administering to said subject a nucleic acid (including modified forms thereof) consisting essentially of a polyT or polyU sequence as well as administering at least one agent that inhibits the production or activity of an immunosuppressant expressed in said tumors.

In other aspects, the invention is directed to compositions containing the foregoing components as well as compounds wherein said components are non-covalently asserted or covalently coupled.

### MODES OF CARRYING OUT THE INVENTION

In order to avoid constantly using terms such as “at least one” or “one or more” that complicate the choice of following verbs, the terms “a” or “an” are defined to include not only a single referent but multiple referents. Thus reference to “an” inhibitory agent effectively means “one or more” or “at least one” inhibitory agent, reference to “a” nucleic acid refers to one or more embodiments of a nucleic acid or “at least one” such embodiments. Further, if an initial reference is made to “a” or “an” composition or method, subsequent referrals to “said” or “the” method or composition also includes plural embodiments.

One component of the compositions, methods and compounds of the invention is a nucleic acid (or modified form thereof) that specifically hybridizes to the polyA tail of a messenger RNA. As noted in the cited US2010/0144831 publication, this can be supplied either in the form of a polyT or polyU sequence-containing nucleic acid, or polyU RNA can be generated using a recombinant vector. The modifications of the nucleic acid are typically those that modify the sugar-phosphate backbone including, for example, the use of phosphorothioate linkages, phosphoramidate linkages, reduced phosphate linkages, peptide nucleic acids, and other modified backbone forms known in the art. Molecules with modified sugars are also included. Modifications to the base sequence are perhaps more challenging as the essentially polyT or polyU character must be maintained. However, alternate forms of the requisite bases may be used as long as their hydrogen bonding patterns match those of polyT or polyU. Of course, nucleic acids with combinations of T/U sequences (polyT/U) could be used such as UTTUTTUTUUU.

Thus, by a “nucleic acid” is meant a nucleic acid molecule which is DNA, RNA, peptide nucleic acid, or other modified backbone molecules, including those with modified phosphodiester linkages with a base sequence that specifically binds polyA. The variety of such nucleic acid molecules is well understood by the ordinarily skilled artisan. For ease of referral, the nucleic acid or polyU RNA will be referred to in the specification as “polyT/polyU.”

The other component of the methods, compositions, and compounds of the invention is an agent that inhibits the activity of, or the production of, an immunosuppressant that is present in the subject's tumor cells. Many immunosuppressants are known, including TGF-β (TGF-β1 and TGF-β2) PGE-2, IL-10, Tregs, and CTLA-4. Agents useful to inhibit expression of these immunosuppressants include but are not limited to antisense nucleic acids and siRNA constructs, ribozymes directed specifically to the RNA encoding these agents. Compositions-of-matter that inhibit the activity of these immunosuppressants include antibodies and fragments thereof that bind specifically to them, mimics of these immunosuppressants that bind their receptors, but fail to activate the receptors, and mimics of the receptors that bind the immunosuppressants themselves. The inhibitory agents that are proteins, such as antibodies or any receptor mimics or immunosuppressant mimics that are peptides can be produced recombinantly in situ, and thus the components of the compositions, compounds or methods of the invention may be nucleic acids encoding these inhibitory materials. Agents that bind and inhibit the immunosuppressive agents include aptamers as well.

In one embodiment, the method of the invention may employ a nucleic acid which contains as a portion, an aptamer capable of binding to the immunosuppressive agent and a portion that comprises the polyU/polyT component. In another embodiment, the polyU/polyT component is coupled to an antibody specific for the immunosuppressive agent. In still another embodiment, the polyT/polyU component is coupled to a nucleic acid-based expression system that generates a protein inhibitor of the immunosuppressant. In still another embodiment, the invention method may employ a DNA that generates polyU coupled to DNA that is transcribed into an antisense nucleic acid or siRNA for inhibition of the production of the immunosuppressive agent.

When administered as separate compositions, any combination of immunosuppressive inhibitor (of production or activity) and polyU/polyT may be used. Each component will be formulated according to standard methods for formulation of such components.

If the components are coupled covalently, this coupling may be through a linker. Bifunctional linkers capable of linking nucleic acids with additional nucleic acids or with small molecules are known. If the entire covalently linked composition is a nucleic acid, the linker may be additional nucleic acid sequence or no linker sequence may be needed.

“Antibodies” are defined as including not only complete immunoglobulins, but also immunoreactive portions thereof, such as Fab or F(ab)₂ portions as well as recombinantly produced single chain Fv antibodies and the like. Many forms of “antibodies” that may be prepared recombinantly are now known in the art. The antibodies may be designed so as not to generate an immune response in a subject of a particular species. Thus, for administration to humans, the antibodies may be humanized or may be fully human as prepared, for example, in the XenoMouse®.

If the components of the methods, compositions, and compounds of the invention are covalently coupled, they may be directly joined, or may be coupled to a bifunctional linker such as a linker containing, for example, groups reactive with amino groups, carboxyl groups, sulfhydryl groups, and the like. If both components of the compositions are nucleic acids, they may be noncovalently associated through overlapping complementary regions.

The possibilities for a combination of inhibitory agent with polyT/polyU are multiple since many manifestations of the inhibitory agents can be used in the alternative and the manner of linkage will depend on the nature of the agent as well as the nature of the nucleic acid molecule containing polyT/polyU. In addition, more than one inhibitory agent may be included in the method, or in the same composition, or in the same molecule. Similarly, more than one particular polyT/polyU embodiment may also be included.

In the method of the invention, the polyU/polyT component and the inhibitory agent may be administered together from separate compositions or in the same composition. They may also be administered sequentially in any order. Administration is preferably intratumoral as the agents and especially the polyT/polyU are universal in their activity. Alternatively, they may be administered as separate components or as a conjugate systematically, for example, by parenteral injection or by transmucosal or transdermal delivery. In these circumstances, it is preferred that the components of the composition be targeted to the tumor, for example, by coupling to a tumor-specific moiety such as an antibody to a tumor-associated antigen. If the components are administered separately, one or both of them may be targeted. In one embodiment, the inhibiting agent(s) may be administered parenterally or otherwise systemically and the polyU/polyT administered intratumorally.

In addition to the required components of the methods, compositions and compounds of the invention, additional chemotherapeutic agents or other antitumor treatment, such as radiation or surgery may be used in combination therewith.

The pharmaceutical compositions will employ standard pharmaceutically acceptable excipients including particulate carrier vehicles such as liposomes, micelles, nanoparticles and the like. PEGylated forms may also be used. Methods and components for preparing pharmaceutical compositions suitable for the active ingredients employed in the treatments of the invention include those found in Remington's Pharmaceutical Sciences, latest edition, Mack Publishing Co., Easton, Pa., incorporated herein by reference. Adapting suitable formulations to the active ingredients is well within the skill of the art. The dosage amounts and regimens are readily designed by the practitioner in light of the specific agent selected for inhibition and the condition of the subject.

The subjects to which the methods of the invention may be applied and the compositions of the invention administered include animals, including mammals, marsupials, fish, and avian species that are subject to developing tumors. The subjects may include laboratory model systems for testing and confirming the efficacy of the methods and optimizing the protocols, such as mice, rats, rabbits, and guinea pigs. The subjects may also be primates, including humans.

1. A method to effect antitumor treatment in a subject, which method comprises administering to a subject in need of such treatment effective amounts of a nucleic acid that contains a nucleotide sequence consisting essentially of polyU, polyT or polyU/T and an inhibitory agent that inhibits the production or activity of an immunosuppressant produced by tumor cells of said subject.

2. The method of claim 1 wherein said nucleic acid and said inhibitory agent are administered in the same composition.

3. The method of claim 1 wherein said nucleic acid and said inhibitory agent are administered sequentially or simultaneously as separate compositions.

4. The method of claim 1 wherein the nucleic acid and the inhibitory agent are conjugated covalently or noncovalently.

5. The method of claim 4 wherein the nucleic acid and the inhibitory agent are coupled covalently directly or through a linker.

6. The method of claim 1 wherein the inhibitory agent inhibits the production or activity of TGF-β1, TGF-β2, PGE-2, IL-10, Tregs or CTLA-4.

7. The method of claim 1 wherein the agent is an antibody, a ribozyme, an interfering RNA, an antisense nucleic acid, a mimic of said immunosuppressant, or a mimic of the receptor for said immunosuppressant.

8. A composition for conducting the method of claim 1.

9. A composition of matter which comprises the nucleic acid and agent of claim 1 as a covalently or noncovalently bound conjugate.

10. A compound which is a conjugate of the nucleic acid and the agent of claim 1.
